(19) United States
(12) Patent Application Publication
(10) Pub. No.: US 2025/0319059 A1
(43) Pub. Date: Oct. 16, 2025

(54) Method for Treating Inflammatory Dysfunction of the Oral Mucosa

(71) Applicant: VIVASOR, INC., San Diego, CA (US)

(72) Inventors: Alexis Nahama, San Diego, CA (US); Alvaro Cisternas, San Diego, CA (US)

(21) Appl. No.: 18/869,950

(22) PCT Filed: May 31, 2023
(86) PCT No.: PCT/US23/23993

§ 371 Date: Nov. 27, 2024

Related U.S. Application Data
(60) Provisional application No. 63/347,285, filed on May 31, 2022.

(51) Int. Cl. A61K 31/357; A61K 45/06; A61K 47/10; A61K 47/26; A61P 1/02; A61P 29/00

(52) U.S. Cl. CPC A61K 31/357; A61K 45/06; A61K 47/10; A61K 47/26; A61P 1/02; A61P 29/00

(57) ABSTRACT

Provided herein, inter alia, is a method for treating inflammatory dysfunction of the oral mucosa comprising topically administering an effective amount of resiniferatoxin (RTX).

This application claims the benefit of priority of U.S. Provisional Patent Application No. 63/347,285, filed May 31, 2022, which is incorporated herein by reference in its entirety for all purposes.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure provides a method for treating inflammatory dysfunction of the oral mucosa comprising topically administering an effective amount of resiniferatoxin (RTX).

### BACKGROUND

RTX acts as an ultrapotent analog of capsaicin, the pungent principal ingredient of the red pepper. RTX is a tricyclic diterpene isolated from certain species of Euphorbia. A homovanillyl group is an important structural feature of capsaicin and is the most prominent feature distinguishing resiniferatoxin from typical phorbol-related compounds. Native RTX has the following structure:

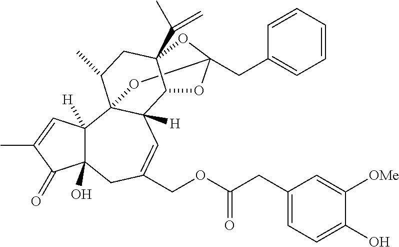

RTX and analog compounds such as tinyatoxin and other compounds (20-homovanillyl esters of diterpenes such as 12-deoxyphorbol 13-phenylacetate 20-homovanillate and mezerein 20-homovanillate) are described in U.S. Pat. Nos. 4,939,194; 5,021,450; and 5,232,684. Other resiniferatoxin-type phorboid vanilloids have also been identified (Szallasi et al. (1999) Brit. J. Pharmacol. 128:428-434).

RTX is known as a TRPVI agonist. TRPV1, the transient receptor potential cation channel subfamily V member 1 (also known as Vanilloid receptor-1 (VR1)) is a multimeric cation channel prominently expressed in nociceptive primary afferent neurons (Caterina et al. (1997) Nature 389:816-824; Tominaga et al. (1998) Neuron 21:531-543). Activation of TRPV1typically occurs at the nerve endings via application of painful heat and is up regulated during certain types of inflammatory stimuli. Activation of TRPV1 in peripheral tissues by a chemical agonist results in the opening of calcium channels and the transduction of a pain sensation (Szalllasi et al. (1999) Mol. Pharmacol. 56:581-587). However, direct application of certain TRPV1 agonists to the cell body of a neuron (ganglion) expressing TRPV1 opens calcium channels and triggers a cascade of events leading to programmed cell death (“apoptosis”) (Karai et al. (2004) J. of Clin. Invest. 113:1344-1352).

Transient receptor potential vanilloid 1 (TRPV1) is reported to be involved in inflammation (Amaya, F. et al. (2003) Brain Res. 963:190-196), cancer (Asai, H. et al. (2005) Pain 117:19-29; Shinoda, M. et al. (2008) J Pain 9:687-699) and neuropathic pain (Rashid, M. et al. (2003) J Pharmacol. Exp. Ther. 304:940-948). In orofacial pain, TRPVI is reported to be involved in tooth pulp inflammation (Tarsa, L. et al. (2010) Neuroscience 167:1205-1215), temporomandibular disorders (TMD) (Ro, J. et al. (2009) Pain 144:270-277), oral cancer (Nagamine, K. et al. (2006) Pain 7:659-670) and IAN injury pain (Kim, H. et al. (2008) J Pain 9:280-288).

Inflammatory dysfunction of the oral mucosa is associated with inflammation and pain of the oral mucosa. Inflammatory dysfunction of the oral mucosa in humans can be attributed to an array of diseases some of which are immune mediated e.g., Bechet disease, burning mouth syndrome, oral lichen planus, pemphigus and pemphigoid, recurrent aphthous stomatitis, stomatitis, Sjogren's syndrome and many others. These diseases tend to be chronic and often severely affect an individual's quality of life. Common causes of inflammatory dysfunction of the oral mucosa include, for example, viral infections, yeast infections, bacterial infections, chemotherapy or radiotherapy treatment for cancer, weakened or deficient immune system, and many more. People with oral mucosal diseases may develop painful mouth sores or ulcers on the mucous membrane lining (the “skin” inside the mouth including cheeks and lips).

Inflammatory dysfunction of the oral mucosa in cats can be attributed to feline chronic gingivostomatitis (FCGS) or caudal stomatitis, and affects between 0.7% and 12% of all cats. FCGS is a debilitating oral inflammatory disease that can last for years leading to euthanasia. FCGS can be multifactorial (arising from viral infection, dental disease and/or hypersensitivity), and can affect gingival, buccal, palatal, sublingual and pharyngeal tissue. FCGS can be refractory to medical (immunosuppression therapy) and surgical management (full mouth dental extraction).

Pain associated with inflammatory dysfunction of the oral mucosa is typically considered to be deep pain requiring interventions which are systemic or direct topical treatments or invasively or surgically directed treatments at the site of pain. The treatments have included topical solutions (applied directly to the gums and teeth for tooth pain), local injections (into gums for tooth pain or into trigger points), a variety of systemic analgesics (aspirin, acetaminophen, non-steroidal anti-inflammatory agents and narcotics), a variety of other systemic agents (steroids, diphenylhydantoin, carbamazepine, calcium channel blockers, beta-blockers, and tricyclic antidepressants) and surgical procedures (tooth extractions, etc.).

Although some treatments for pain associated with inflammatory dysfunction of the oral mucosa are available, they are not always effective. Moreover, though some treatments are effective in treating the pain itself (for example, U.S. Pat. No. 5,296,225 and US patent publication No. U.S. Pat. No. 20,150,051271), none are capable of treating the underlying neurogenic inflammation.

Accordingly, there is a need in the art for improved treatment for pain and inflammation associated with inflammatory dysfunction of the oral mucosa which would address not only sensitivity to pain but the underlying neurogenic inflammation causing such pain.

### SUMMARY

The present disclosure aims to meet this need and/or provide other benefits. Provided herein are methods of administering RTX topically for treatment of inflammatory dysfunction of the oral mucosa to a subject in need thereof. This disclosure is based in part on the realization that oral topical administration of RTX to treat inflammatory dysfunction of the oral mucosa can provide effective pain relief as well as treat the underlying neurogenic inflammation.

In an aspect, provided herein is a method for treating an inflammatory dysfunction of the oral mucosa, comprising topically administering to a subject in need of treatment of the inflammatory dysfunction of the oral mucosa a therapeutically effective amount of resiniferatoxin (RTX).

In an aspect, provided herein is a composition comprising resiniferatoxin (RTX) for use in a method for treating an inflammatory dysfunction of the oral mucosa, the method comprising topically administering to a subject in need of treatment of the inflammatory dysfunction of the oral mucosa a therapeutically effective amount of said composition.

In embodiments, the inflammatory dysfunction of the oral mucosa is associated with neurogenic inflammation. In embodiments, the inflammatory dysfunction of the oral mucosa is associated with pain.

In embodiments, the neurogenic inflammation is reduced. In embodiments, the pain is reduced.

In embodiments, the pain is orofacial pain. In embodiments, the pain is attributed to disorders of dentoalveolar structures, cancer, neuropathic pain, or idiopathic pain. In embodiments, the pain is attributed to disorders of dentoalveolar structures. In embodiments, the pain is attributed to cancer. In embodiments, the pain is attributed to neuropathic pain. In embodiments, the pain is attributed to idiopathic pain. In embodiments, the pain is attributed to stomatitis. In embodiments, the pain is attributed to chronic gingivostomatitis (CGS). In embodiments, the pain is attributed to feline chronic gingivostomatitis (FCGS). In embodiments, the idiopathic pain is attributed to burning mouth syndrome.

In embodiments, the pain is attributed to radiotherapy or chemotherapy. In embodiments, the pain is attributed to radiotherapy. In embodiments, the pain is attributed to chemotherapy. In embodiments, the pain is attributed to radiotherapy induced mucositis.

In embodiments, the pain is attributed to disorders of dentoalveolar structures. In embodiments, the pain is attributed to dental pain or oral mucosal pain. In embodiments, the pain is attributed to dental pain. In embodiments, the pain is attributed to oral mucosal pain.

In embodiments, the subject is a mammal. In embodiments, the mammal is a cat, dog, horse, pig, ruminant, cow, sheep, goat, or domesticated mammal. In embodiments, the mammal is a cat. In embodiments, the mammal is a dog. In embodiments, the mammal is a horse. In embodiments, the mammal is a pig. In embodiments, the mammal is a ruminant. In embodiments, the mammal is a cow. In embodiments, the mammal is a sheep. In embodiments, the mammal is a goat. In embodiments, the mammal is a domesticated mammal. In embodiments, the mammal is a human.

In embodiments, the RTX is administered in a pharmaceutical formulation comprising the RTX and a pharmaceutically acceptable carrier. In embodiments, the pharmaceutically acceptable carrier comprises water. In embodiments, the pharmaceutically acceptable carrier comprises polysorbate 80. In embodiments, the pharmaceutically acceptable carrier comprises polyethylene glycol. In embodiments, the pharmaceutically acceptable carrier comprises a sugar or sugar alcohol. In embodiments, the pharmaceutically acceptable carrier comprises mannitol. In embodiments, the pharmaceutically acceptable carrier comprises dextrose. In embodiments, the pharmaceutically acceptable carrier comprises a pharmaceutically acceptable buffer.

In embodiments, the pharmaceutically acceptable buffer is phosphate buffer and/or the pH of the formulation is about 6.0-7.6 or about 7.2. In embodiments, the pharmaceutically acceptable carrier comprises a pharmaceutically acceptable salt. In embodiments, the pharmaceutically acceptable salt is NaCl.

In embodiments, the RTX is administered in a dose of from about 0.1 μg to about 100 μg. In embodiments, the concentration of RTX in the pharmaceutical formulation is in the range of 0.02 to 300 g/ml. In embodiments, the concentration of RTX in the pharmaceutical formulation is in the range of 0.02-0.1 μg/ml, 0.1-1 μg/ml, 1-5 μg/ml, 5-10 μg/ml, 10-20 μg/ml, 20-50 μg/ml, 50-100 μg/ml, 100-150 μg/ml, 150-200 μg/ml, 200-250 μg/ml, or 250-300 μg/ml. In embodiments, the concentration of RTX in the pharmaceutical formulation is in the range of 0.1-50 μg/ml.

In embodiments, the RTX is topically administered in a volume of 0.5-5 ml. In embodiments, the RTX is topically administered in a volume in the range of 0.5-1.0 ml, 1.0-1.5 ml, 1.5-2 ml, 2-3 ml, 3-4 ml, or 4-5 ml.

In embodiments, a general or a local anesthetic is administered prior to administration of RTX. In embodiments, an analgesic is administered following administration of RTX. In embodiments, the analgesic is an opioid or a nonsteroidal anti-inflammatory drug (NSAID). In embodiments, the analgesic is an opioid. In embodiments, the analgesic is a nonsteroidal anti-inflammatory drug (NSAID). In embodiments, the RTX is administered to a plurality of sites.

In an aspect, provided herein is a method for treating a neurogenic inflammation associated with an inflammatory dysfunction of the oral mucosa, comprising topically administering to a subject in need of treatment of the neurogenic inflammation a therapeutically effective amount of resiniferatoxin (RTX).

In an aspect, provided herein is a composition comprising resiniferatoxin (RTX) for use in a method for treating a neurogenic inflammation associated with an inflammatory dysfunction of the oral mucosa, the method comprising topically administering to a subject in need of treatment of the neurogenic inflammation associated with an inflammatory dysfunction of the oral mucosa a therapeutically effective amount of said composition.

### FIGURES

FIG. 1A Shows body weight of each cat at the beginning of the study (day 0) and at the conclusion of the study (day 8).

FIG. 1B Shows each cat's food consumption as % ration consumed over the eight days of the study (there was no data collection on day 2).

FIG. 2A-E Time dependent VSOM score demonstrates the effectiveness of 6.25 μg, 12.5 μg, and 25 μg RTX treatment (qualitative assessment). FIG. 2A Time dependent VSOM score demonstrates the effectiveness of 6.25 μg RTX treatment. FIG. 2B Time dependent VSOM score demonstrates the effectiveness of 12.5 μg RTX treatment. FIG. 2C Time dependent VSOM score demonstrates the effectiveness of 25 μg RTX treatment. FIG. 2D Time dependent VSOM score (by groups) demonstrates the effectiveness of 6.25 μg, 12.5 μg, and 25 μg RTX treatment (qualitative assessment). FIG. 2E. Graph of % Maximum possible effect (% MPE) shows % change in VSOM from day 0 (before treatment) to day 28, demonstrating the effectiveness of 6.25 μg, 12.5 μg, and 25 μg RTX treatment (qualitative assessment).

FIG. 3A Effectiveness of 6.25 μg, 12.5 μg, and 25 μg RTX treatment is demonstrated via delta in SDAI score of each cat (semi-quantitative assessment).

FIG. 3B Graph of % Maximum possible effect (% MPE) shows % change in SDAI from day 0 (before treatment) to day 28 for each cat, demonstrating the effectiveness of 6.25 μg, 12.5 μg, and 25 μg RTX treatment (semi-quantitative assessment).

FIG. 3C Shows the baseline SDAI score for each cat in three treatment groups (6.25 μg, 12.5 μg, and 25 μg RTX).

FIG. 4 Effectiveness of 6.25 μg, 12.5 μg, and 25 μg RTX treatment is demonstrated via SDAI scores on day 0 (before treatment) and day 28, for each cat.

### DETAILED DESCRIPTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like. It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, terms “mcg”, “μg”, and “ug” are interchangeable and refer to micrograms.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., +10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In embodiments, “about” encompasses variation within 10%, 5%, 2%, 1%, or 0.5% of a stated value.

Numeric ranges are inclusive of the numbers defining the range. Measured and measurable values are understood to be approximate, taking into account significant digits and the error associated with the measurement. Also, all ranges are to be interpreted as encompassing the endpoints in the absence of express exclusions such as “not including the endpoints”; thus, for example, “ranging from 1 to 10” includes the values 1 and 10 and all integer and (where appropriate) non-integer values greater than 1 and less than 10.

The use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings. Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

The term “inflammatory dysfunction of the oral mucosa” as used herein refers to inflammation and pain associated with oral mucosal diseases, some of which are immune mediated, e.g., Behcet disease, burning mouth syndrome, oral lichen planus, pemphigus and pemphigoid, recurrent aphthous stomatitis, Sjogren's syndrome, caudal stomatitis, stomatitis, chronic gingivostomatitis (CGS), feline chronic gingivostomatitis (FCGS), or oral cancer. These diseases tend to be chronic and often severely affect an individual's quality of life. Common causes of inflammatory dysfunction of the oral mucosa include, for example, viral infections, yeast infections, bacterial infections, chemotherapy or radiotherapy treatment for cancer, weakened or deficient immune system, and many more. Mammals with oral mucosal diseases may develop painful mouth sores or ulcers on the mucous membrane lining (the “skin” inside the mouth including cheeks and lips).

The term “orofacial pain” as used herein refers to pain arising from disorders of dentoalveolar structures e.g., dental pain or oral mucosal pain. The oral mucosal pain may be attributed to neurogenic inflammation (which can be a result of various oral mucosal diseases). The dental pain may be a result of pulpal pain, periodontal pain or gingival pain. “orofacial pain” as used herein also refers to pain arising from idiopathic oral pain e.g. burning mouth syndrome or persistent idiopathic dentoalveolar pain. “orofacial pain” as used herein may also refer to neuropathic pain or pain arising from local or systemic inflammation. Additionally, the term “orofacial pain” as used herein refers to pain attributed from cancer (e.g. oral cancer) or from cancer treatment, for example, radiotherapy. In embodiments, orofacial pain may be attributed to radiotherapy or chemotherapy induced mucositis. In embodiments, orofacial pain may be attributed to stomatitis. In embodiments, orofacial pain may be attributed to chronic gingivostomatitis.

The term “neuropathic pain” refers to pain that results from damage or disease affecting sensory neurons.

A “ruminant” is a mammal that has a rumen. Examples of ruminants include, but are not limited to cattle, sheep, antelopes, deer, and giraffes.

The terms “effective amount”, “therapeutically effective amount” or “effective dose” or related terms may be used interchangeably and refer to an amount of the therapeutic agent (RTX) that when administered to a subject, is sufficient to affect a measurable improvement or even complete resolution of the pain and neurogenic inflammation associated with inflammatory dysfunction of the oral mucosa. For example, administering an effective dose sufficient to inhibit neurogenic inflammation and stop the pain in a subject. Therapeutically effective amounts of the therapeutic agent (RTX) provided herein, will vary depending upon the subject and disease condition being treated, the weight of the subject, the severity of the disease condition in the subject, and the like, which can readily be determined by one of ordinary skill in the art. In one embodiment, a therapeutically effective amount will depend on certain aspects of the subject to be treated and the disorder to be treated and may be ascertained by one skilled in the art using known techniques. In addition, as is known in the art, adjustments for body weight, general health, time of administration, drug interaction, and the severity of the disease may be necessary.

The terms “subject” and “patient” as used herein refer to human and non-human animals, including vertebrates, mammals and non-mammals. In one embodiment, the subject can be human, non-human primate, simian, ape, murine (e.g., mice and rats), bovine, porcine, equine, canine, feline, caprine, lupine, ranine or piscine.

The term “administering”, “administered” and grammatical variants refers to the physical introduction of a therapeutic agent to a subject, using any of the various methods and delivery systems known to those skilled in the art. Exemplary routes of administration for the formulations disclosed herein include a non-parenteral route (i.e., local), e.g., topical, epidermal or mucosal route of administration. In one embodiment, the formulation is administered topically to the oral mucosa in the mouth in the form of solution, suspension, cream, lotion, gel, paste, spray, powder, or ointment. Administering can also be performed, for example, once, a plurality of times, and/or over one or more extended periods.

“Treating” is to be understood broadly and encompasses any beneficial effect, including, e.g., delaying, slowing, or arresting the worsening of symptoms associated with inflammatory dysfunction of the oral mucosa or remedying such symptoms, at least in part. Treating also encompasses bringing about any form of improved patient function, as discussed in detail below. In embodiments, treatment also means prolonging survival as compared to expected survival if not receiving treatment. The term is also meant to include alleviating or abrogating a disorder, disease, or condition, or one or more of the symptoms associated with the disorder, disease, or condition; or alleviating or eradicating the cause(s) of the disorder, disease, or condition itself. Those in need of treatment include those who already have the disease or disorder.

A “pharmaceutically acceptable vehicle” for therapeutic purposes is a physical embodiment that can be administered to a subject. Pharmaceutically acceptable vehicles include solutions, suspensions, creams, lotions, gels, pastes, sprays, powders, or ointments, but is not limited to these. An example of a pharmaceutically acceptable vehicle is a buffered isotonic solution such as phosphate buffered saline (PBS).

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Well-tolerated” as used herein refers to a response to administration of RTX in which the subject experiences little to no clinical adverse effects (e.g., only mild or moderate adverse effects, or only mild adverse effects). Mild adverse effect (e.g., minor event requiring no specific medical intervention; asymptomatic laboratory findings only; marginal clinical relevance); Moderate adverse effect (e.g., event requiring only minimal intervention; local intervention; non-invasive intervention; transfusion; elective interventional radiological procedure; or therapeutic endoscopy or operation). Adverse effects include unfavorable and unintended signs, symptoms, or diseases associated with the use of RTX. Examples of adverse effects include but are not limited to toxicity and distress indicated by, for example, high or low body temperature, hypotension, hypertension, hypocarbia, and ventricular arrhythmias, lethargy, responsiveness, loss of appetite, and/or weight loss.

### Overview

In chronic pain states, vanilloid (TRPV1) receptors are up regulated on neurons, have reduced stimulation thresholds, and cause an increased perception of pain. TRPV1 agonists, such as capsaicin, will activate and depolarize TRPV1 receptors, initially causing a burning sensation from stimulation of the nerve. After the TRPV1 receptors are completely depolarized, the nociceptive areas are desensitized and become analgesic, particularly to neuropathic pain (Bley, K. (2010) TRPV1 agonist approaches for pain management. In: Gomtsyan A, Faltynek CR, eds. Vanilloid Receptor TRPV1 in Drug Discovery: Targeting Pain and Other Pathological Disorders. New York: Wiley, 325-47). Capsaicin, a less potent TRPV1 agonist than RTX, has been approved for the treatment of postherpetic neuralgia (Acorda Therapeutics, 2009). RTX, the most potent TRPV1 receptor agonist, is 1000-10,000 times more potent than capsaicin or 16 billion on the Scoville scale

Vanilloid receptor-1 (TRPV1) is a multimeric cation channel prominently expressed in nociceptive primary afferent neurons (see, e.g., Caterina et al., (1997) Nature 389:8160824; Tominaga et al., (1998) Neuron 531-543). Activation of the receptor typically occurs at the nerve endings via application of painful heat (TRPV1 transduces heat pain) or during inflammation or exposure to vanilloids. It has been widely reported that activation of TRPV1-expressing afferents causes secretion of neuropeptides such as substance P (SP) and calcitonin gene-related peptide (CGRP) (See Nicoletti et al. (2012) Int J Immunopathol Pharmacol 25 (4): 849-57; Bhatia (2010) Antioxid Redox Signal 12 (10): 1191-202; Fernandes et al. (2009) Handb Exp Pharmacol 194:393-416; Scardina et al. (2004) Minerva Stomatol 53 (1-2): 21-32; Harrison et al. (2001) Int J Biochem Cell Biol 33 (6): 555-76). Released SP, but not CGRP, in sensory endings binds neurokinin (NK) 1 receptors on blood vessels causes vasodilation and increased vascular permeability that allows loss of proteins and fluid (plasma extravasation) thus promoting the regional accumulation of monocytes and leukocytes contributing to inflammation (See Roberts et al. (2004) Brain Res 995 (2): 176-83; Andrews et al. (1989) Br J Pharmacol 97 (4): 1232-8; and McConalogue et al. (1998) Mol Biol Cell 9 (8): 2305-24).

Activation of TRPVI by an agonist, such as resiniferatoxin or capsaicin, results in the opening of calcium channels and the transduction of pain sensation (see, e.g., Szalllasi et al., (1999) Mol. Pharmacol. 56:581-587). After an initial activation of TRPV1, TRPV1 agonists desensitize TRPV1 to subsequent stimuli. This desensitization phenomenon has been exploited in order to produce analgesia to subsequent nociceptive challenge.

It has been shown that topical administration of resinferatoxin (RTX), which is a potent vanilloid receptor agonist, at the nerve endings in the skin triggers a long-lasting insensitivity to chemical pain stimulation (for example: US publication US20210007998; US patent U.S. Pat. No. 5,296,225). Such topical application, however, is limited to the surface of the skin and only addresses the sensation of pain but not the underlying neurogenic inflammation.

The present disclosure provides topical formulations of RTX which can be applied to the oral mucosa or oral cavity, and which can treat not only the pain associated with inflammatory dysfunction of the oral mucosa but the underlying neurogenic inflammation, affecting gingival, buccal, palatal, sublingual or pharyngeal tissue, causing said pain.

### Exemplary Methods and Compositions for Use

Provided herein are topical formulations of RTX for application to the oral mucosa or oral cavity for control or reduction of pain associated inflammatory dysfunction of the oral mucosa and underlying neurogenic inflammation.

In an aspect, provided herein is a method for treating an inflammatory dysfunction in the oral mucosa, comprising topically administering to a subject in need of treatment of the inflammatory dysfunction of the oral mucosa a therapeutically effective amount of RTX.

In another aspect, provided herein is a composition comprising RTX for use in a method for treating an inflammatory dysfunction of the oral mucosa, the method comprising topically administering to a subject in need of treatment of the inflammatory dysfunction of the oral mucosa a therapeutically effective amount of said composition.

In yet another aspect, provided herein are methods and compositions for interrupting a neurogenic inflammatory process occurring in the mouth and/or treating neurogenic inflammation associated with an inflammatory dysfunction of the oral mucosa, comprising topically administering to a subject a therapeutically effective amount of RTX.

In yet another aspect, provided herein is a method for treating neurogenic inflammation associated with an inflammatory dysfunction of the oral mucosa, comprising topically administering to a subject in need of treatment of the neurogenic inflammation a therapeutically effective amount of RTX.

In embodiments, the subject had one or more symptoms of inflammatory dysfunction of oral mucosa prior to treatment and the treatment reduces or abrogates one or more symptoms. For example, symptoms of inflammatory dysfunction of oral mucosa include orofacial pain, neurogenic inflammation, idiopathic pain, neuropathic pain, mouth ulcers or sores, blisters, or discomfort when eating.

The compositions and methods described herein are for use with any subject in whom RTX is effective, e.g., able to bind and activate TRPV-1 or a homolog thereof, and who is in need of treatment for inflammatory dysfunction of the oral mucosa. In embodiments, the subject is a mammal. In embodiments, the mammal is a human. In embodiments, the mammal is a cat. In embodiments, the mammal is a dog. In embodiments, the mammal is a monkey. In embodiments, the mammal is a ruminant. In embodiments, the mammal is a horse, cow, pig, sheep, goat, or domesticated mammal.

The compositions and methods described herein are for use with any subject in whom RTX is effective, e.g., able to bind and activate TRPV-1 or a homolog thereof, and who is in need of treatment for neurogenic inflammation.

### Administration and Formulations

RTX may be administered topically to the oral mucosa or the oral cavity. RTX may be administered topically to one or more than one site, depending on the number of inflammation sites inside the oral cavity or on the oral mucosa. In embodiments, the RTX is administered topically to a single site. In embodiments, the RTX is administered topically to more than one site. In embodiments, the RTX is administered to a plurality of sites.

In embodiments, the entire dose of RTX is topically applied to a site in a single application. In embodiments, the entire dose of RTX is topically applied to a site in more than one application. In embodiments, the entire dose of RTX is topically applied to a site in two applications. In embodiments, the entire dose of RTX is topically applied to a site in three applications. In embodiments, the entire dose of RTX is topically applied to a site in four applications. In embodiments, the entire dose of RTX is topically applied to a site in five applications. In embodiments, each lesion in the oral cavity received one coat of RTX. In embodiments, each lesion in the oral cavity received two coats of RTX. In embodiments, each lesion in the oral cavity received three coats of RTX. In embodiments, each lesion in the oral cavity received four coats of RTX. In embodiments, each lesion in the oral cavity received five coats of RTX. In embodiments, the RTX solution was allowed to drip into the gingival sulcus.

In embodiments, the method further comprises administering a general or a local anesthetic prior to administration of RTX. In embodiments, the method further comprises administering a general anesthetic prior to administration of RTX. In embodiments, the method further comprises administering a local anesthetic prior to administration of RTX.

In embodiments, the subject is placed under anesthesia before administration of RTX. In embodiments, the local anesthetic is administered via injection. In embodiments, the local anesthetic is administered topically.

In embodiments, the local anesthetic is an amino-amide anesthetic, such as lidocaine, mepivacaine, prilocaine, bupivacaine, etidocaine, ropivacaine, or levobupivacaine. In embodiments, the topical local anesthetic is benzocaine, lidocaine, cocaine, proparacaine, or oxybuprocaine.

Post treatment discomfort can be managed with analgesics. In embodiments, the method further comprises administering an analgesic following administration of RTX. In embodiments, the analgesic is an opioid or a nonsteroidal anti-inflammatory drug (NSAID). In embodiments, the analgesic is a nonsteroidal anti-inflammatory drug (NSAID). In embodiments, the analgesic is an opioid. In embodiments, the opioid is Buprenorphine.

In embodiments, the analgesic administered, following administration of RTX, for no more than 5 days. In embodiments, the analgesic administered, following administration of RTX, for no more than 4 days. In embodiments, the analgesic administered, following administration of RTX, for no more than 3 days. In embodiments, the analgesic administered, following administration of RTX, for no more than 2 days. In embodiments, the analgesic administered, following administration of RTX, for no more than 1 day. In embodiments, no analgesic is administered following administration of RTX.

In embodiments, for topical administration, RTX can be formulated into solutions, lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like.

In embodiments, the pharmaceutical formulations provided herein are formulated as a cream for topical administration, which comprise RTX and one or more pharmaceutically acceptable excipients or carriers. In one embodiment, the cream provided herein comprises RTX and one or more excipients or carriers selected from the group consisting of water, octyldodecanol, mineral oil, stearyl alcohol, cocamide DEA, polysorbate 80, myristyl alcohol, sorbitan monostearate, lactic acid, and benzyl alcohol. In another embodiment, the cream provided herein comprises RTX and water, octyldodecanol, mineral oil, stearyl alcohol, cocamide DEA, polysorbate 80, myristyl alcohol, sorbitan monostearate, lactic acid, and benzyl alcohol.

In embodiments, the pharmaceutical formulations provided herein are formulated as a gel for topical administration, which comprise RTX and one or more pharmaceutically acceptable excipients or carriers. In one embodiment, the gel provided herein comprises RTX and one or more excipients or carriers selected from the group consisting of water, isopropyl alcohol, octyldodecanol, dimethicone copolyol 190, carbomer 980, sodium hydroxide, and docusate sodium. In another embodiment, the gel provided herein comprises RTX and water, isopropyl alcohol, octyldodecanol, dimethicone copolyol 190, carbomer 980, sodium hydroxide, and docusate sodium.

In embodiments, the pharmaceutical formulations provided herein are formulated as a solution for topical administration, which comprise RTX and one or more pharmaceutically acceptable excipients or carriers.

In embodiments, the RTX, is administered with a pharmaceutically acceptable carrier. In embodiments, the pharmaceutically acceptable carrier comprises water. In embodiments, the pharmaceutically acceptable carrier comprises polysorbate 80. In embodiments, the pharmaceutically acceptable carrier comprises polyethylene glycol. In embodiments, the pharmaceutically acceptable carrier comprises sugar or sugar alcohol. In embodiments, the pharmaceutically acceptable carrier comprises mannitol. In embodiments, the pharmaceutically acceptable carrier comprises dextrose. In embodiments, the pharmaceutically acceptable carrier comprises a pharmaceutically acceptable buffer. In embodiments, the pharmaceutically acceptable carrier comprises a phosphate buffer. In embodiments, the pharmaceutically acceptable carrier comprises a pharmaceutically acceptable salt. In embodiments, the pharmaceutically acceptable carrier comprises NaCl. In embodiments, the pharmaceutically acceptable carrier comprises an organic solvent such as ethanol or DMSO, e.g., as a minority or residual component used as an aid in dissolving RTX before dilution in a primarily aqueous composition.

The concentration of RTX in the formulation may be any suitable value for delivery of the intended dose. In embodiments, the concentration of RTX in the pharmaceutical formulation is in the range of 0.02 to 300 μg/ml. In embodiments, the concentration of RTX in the pharmaceutical formulation is in the range of 0.02-0.1 μg/ml, 0.1-1 μg/ml, 1-5 μg/ml, 5-10 μg/ml, 10-20 μg/ml, 20-50 μg/ml, 50-100 μg/ml, 100-150 μg/ml, 150-200 μg/ml, 200-250 μg/ml, or 250-300 μg/ml. In embodiments, the concentration of RTX in the pharmaceutical formulation is 0.5 μg/ml-0.6 μg/ml, 0.6 μg/ml-0.7 μg/ml, 0.7 μg/ml-0.8 μg/ml, 0.8 μg/ml-0.9 μg/ml, 0.9 μg/ml-1.0 μg/ml, 1.0 μg/ml-1.1 μg/ml, 1.1 μg/ml-1.2 μg/ml, 1.2 μg/ml-1.3 μg/ml, 1.3 μg/ml-1.4 μg/ml, 1.4 μg/ml-1.5 μg/ml, 1.5 μg/ml-2 μg/ml, 2 μg/ml-3 μg/ml, 3 μg/ml-4 μg/ml, 4 μg/ml-5 μg/ml, 5 μg/ml-6 μg/ml, 6 μg/ml-7 mcg/ml, 7 μg/ml-8 μg/ml, 8 μg/ml-9 μg/ml, 9 μg/ml-10 μg/ml, 10 μg/ml-11 μg/ml, 11 μg/ml-12 μg/ml, 12 μg/ml-13 μg/ml, 13 μg/ml-14 μg/ml, or 14 μg/ml-15 μg/ml. In embodiments, the concentration of RTX in the pharmaceutical formulation is in the range of 0.1-50 μg/ml, 1-50 μg/ml, or 12.5 μg/ml. In embodiments, the concentration of RTX in the pharmaceutical formulation is in the range of 0.1 to 100 μg/ml, such as 0.1 to 50 μg/ml or 1 to 25 μg/ml, or about 0.1, 0.2, 0.5, 1, 1.5, 2, 3, 4, 5, 6, 7, 8, 9, 10, 11, 12, 13, 14, 15, 20, 25, 30, 40, or 50 μg/ml.

In embodiments, the RTX is delivered in a composition having a volume of 0.1 ml-1 ml, 1 ml-10 ml, 10 ml-20 ml, 20 ml-30 ml, 30 ml-40 ml, 40 ml-50 ml, 50 ml-60 ml, 60 ml-70 ml, 70 ml-80 ml, 80 ml-90 ml, or 90 ml-100 ml. In embodiments, the RTX is delivered in a composition having a volume of 0.5 ml-5 ml. In embodiments, the RTX is delivered in a composition having a volume in the range of 0.5-1.0 ml, 1.0-1.5 ml, 1.5-2 ml, 2-3 ml, 3-4 ml, or 4-5 ml.

Starting from a concentrated stock solution, a formulation of RTX for delivery into a subject may be prepared by dilution in an appropriate diluent, such as saline.

The formulation may have any pH suitable for topical administration. In embodiments, the pharmaceutical formulation comprising RTX and a pharmaceutically acceptable carrier has a pH in the range of 6.0 to 7.6. In embodiments, the pharmaceutical formulation comprising RTX and a pharmaceutically acceptable carrier has a pH in the range of 6.0 to 6.4, 6.3 to 6.7, 6.4 to 6.8, 6.8 to 7.2, 7 to 7.4, or 7.2 to 7.6. In embodiments, the pharmaceutical formulation comprising RTX and a pharmaceutically acceptable carrier has a pH of about 6.5 or about 7.2.

In embodiments, the formulation comprises polysorbate 80 and dextrose. In embodiments, the concentration of polysorbate 80 is 0.03-7% w/v. In embodiments, the concentration of polysorbate 80 is 2-4% w/v, and/or the concentration of dextrose is 4-6% w/v. In embodiments, the concentration of polysorbate 80 is 3% w/v, and/or the concentration of dextrose is 5% w/v. The formulation may further comprise a buffer, such as phosphate buffer (e.g., sodium phosphate buffer). In embodiments, the concentration of phosphate buffer is 10-50 mM. In embodiments, the concentration of phosphate buffer is 10-30 mM. In embodiments, the concentration of phosphate buffer is 10 mM. In embodiments, the concentration of phosphate buffer is 30 mM. The formulation may have a pH in the range of 7-7.5, such as about 7.2. In embodiments, in any of the foregoing formulations, the concentration of RTX may be 1-30μg/ml, such as 3.12 μg/ml or 12.5 μg/ml. In embodiments, the formulation further comprises phosphate buffer, e.g., at a concentration and pH shown for phosphate buffer in Table 1. In embodiments, the formulation further comprises NaCl, e.g., at a concentration shown for NaCl in Table 1. When both are present, the phosphate buffer and NaCl may be (but are not necessarily) present at a combination of concentrations and phosphate buffer pH shown for an individual formulation.

Exemplary formulations of RTX are shown in the following table.

TABLE
Exemplary RTX Solution Formulations

| Formu- | | | |
|---|---|---|---|
| lation | | Component | |
| Number | Formulation Components | Concentration | |
| 1 | RTX | 200 | mcg/mL |
| | Polysorbate 80 | 7.0% | w/v |
| | Dextrose | 0.8% | w/v |
| | 30 mM Phosphate Buffer w/0.44% NaCl | 30 mM, pH 7.2 | |
| 2 | RTX | 200 | mcg/mL |
| | Polyethylene Glycol 300 | 3.0% | v/v |
| | Polysorbate 80 | 0.1% | w/v |
| | Dextrose | 0.8% | w/v |
| | 10 mM Phosphate Buffer w/0.73% NaCl | 10 mM, pH 6.5 | |
| 3 | RTX | 200 | mcg/mL |
| | Polyethylene Glycol 300 | 30.0% | v/v |
| | Polysorbate 80 | 1.0% | w/v |
| | 10 mM Phosphate Buffer w/0.86% NaCl | 10 mM, pH 6.5 | |
| 4 | RTX | 200 | mcg/mL |
| | Polyethylene Glycol 300 | 30.0% | v/v |
| | Polysorbate 80 | 0.04% | w/v |
| | 10 mM Phosphate Buffer w/0.88% NaCl | 10 mM, pH 6.5 | |
| 5 | RTX | 200 | mcg/mL |
| | Polysorbate 80 | 3.0% | w/v |
| | Dextrose | 0.8% | w/v |
| | 30 mM Phosphate Buffer w/0.54% NaCl | 30 mM, pH 7.2 | |
| 6 | RTX | 200 | mcg/mL |
| | Polysorbate 80 | 3.0% | w/v |
| | Mannitol | 0.8% | w/v |
| | 30 mM Phosphate Buffer w/0.54% NaCl | 30 mM, pH 7.2 | |
| 7 | RTX | 200 | mcg/mL |
| | Polysorbate 80 | 7.0% | w/v |
| | Mannitol | 0.8% | w/v |
| | 30 mM Phosphate Buffer w/0.45% NaCl | 30 mM, pH 7.2 | |
| 8 | RTX | 200 | mcg/mL |
| | Polyethylene Glycol 300 | 3.0% | v/v |
| | Polysorbate 80 | 0.1% | w/v |
| | Mannitol | 0.8% | w/v |
| | 10 mM Phosphate Buffer w/0.74% NaCl | 10 mM, pH 6.5 | |
| 9 | RTX | 200 | mcg/mL |
| | Polyethylene Glycol 300 | 3.0% | v/v |
| | Polysorbate 80 | 0.1% | w/v |
| | Dextrose | 3.0% | w/v |
| | 10 mM Phosphate Buffer w/0.34% NaCl | 10 mM, pH 6.5 | |
| 10 | RTX | 200 | mcg/mL |
| | Polyethylene Glycol 300 | 3.0% | v/v |
| | Polysorbate 80 | 0.1% | w/v |
| | Mannitol | 3.0% | w/v |
| | 10 mM Phosphate Buffer w/0.36% NaCl | 10 mM, pH 6.5 | |
| 11 | RTX | 200 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 12 | RTX | 200 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 13 | RTX | 25 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 14 | RTX | 25 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 15 | RTX | 100 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 16 | RTX | 200 | mcg/mL |
| | Polysorbate 80 | 7.0% | w/v |
| | Dextrose | 5.0% | w/v |

| | |
|---|---|
| 30 mM Phosphate Buffer w/0.54% NaCl | 30 mM, pH 7.2 |

In embodiments, formulations in Table 1 include dextrose. In embodiments, the concentration of dextrose is 0.05-5% w/v. In embodiments, the concentration of dextrose is 0.8-5% w/v. In embodiments, the concentration of dextrose is 0.05% w/v. In embodiments, the concentration of dextrose is 0.8% w/v. In embodiments, the concentration of dextrose is 3.0% w/v. In embodiments, the concentration of dextrose is 5.0% w/v.

In embodiments, formulations in Table 1 include mannitol. In embodiments, the concentration of mannitol is 0.8-3.0% w/v. In embodiments, the concentration of mannitol is 0.8% w/v. In embodiments, the concentration of mannitol is 3.0% w/v.

In embodiments, the dextrose or mannitol is omitted from a formulation shown in Table 1.

In embodiments, the concentration of RTX in a formulation shown in Table 1 is adjusted to any of the RTX concentrations or concentration ranges disclosed herein. For example, in embodiments, the concentration of RTX in a formulation shown in Table 1 is adjusted to 0.3-200 mcg/ml. In embodiments, the concentration of RTX in a formulation shown in Table 1 is 200 mcg/ml. In embodiments, the concentration of RTX in a formulation shown in Table 1 is 0.3-100 mcg/ml. In embodiments, the concentration of RTX in a formulation shown in Table 1 is 100 mcg/ml. In embodiments, the concentration of RTX in a formulation shown in Table 1 is adjusted to 0.3-50 mcg/ml. In embodiments, the concentration of RTX in a formulation shown in Table 1 is 25 mcg/ml. As another example, in embodiments, the concentration of RTX in a formulation shown in Table 1 is adjusted to 0.3-15 mcg/ml. As another example, in embodiments, the concentration of RTX in a formulation shown in Table 1 is adjusted to 0.5-10 mcg/ml. As another example, in embodiments, the concentration of RTX in a formulation shown in Table 1 is adjusted to 0.6-1.5 mcg/ml. The dextrose or mannitol is omitted from any such formulation having an adjusted RTX concentration.

The formulations in Table 1 may be prepared according to the following exemplary methods, which are provided for formulations 3 and 5 but may be adapted to the other formulations by one skilled in the art. Formulation 3 may be made by adding 46 mg sodium phosphate monobasic monohydrate, 94.7 mg sodium phosphate dibasic anhydrous, and 860 mg NaCl to a 100 ml volumetric flask. 50 ml of water is added to dissolve the components in the flask, followed by addition of 1.0 g of polysorbate 80, to form the aqueous component. 20 mg of RTX is added to the aqueous component in the volumetric flask, and pH is adjusted with hydrochloric acid/sodium hydroxide to 7.2. Then 30 mL of PEG 300 is added, and the solution is sonicated to dissolve the solids. It should be noted that RTX will sometimes precipitate at the interface of aqueous solution and PEG initially but will go back into solution upon sonication. The full mixture in the flask is diluted to volume (100.00 ml) with water and this is mixed by an inversion process. The full formulation is filtered through a 0.2 μm polytetrafluoroethylene (PTFE) filter.

Formulation 5 may be made by adding 138 mg sodium phosphate monobasic monohydrate, 284.1 mg sodium phosphate dibasic anhydrous, and 540 mg NaCl to a 100 ml volumetric flask. 50 ml of water is added to dissolve the components in the flask, followed by addition of 3.0 g of polysorbate 80, and 800 mg of dextrose to form the aqueous component. 20 mg of RTX is added to the aqueous component in the volumetric flask, and pH is adjusted with hydrochloric acid/sodium hydroxide to 7.2. The solution is then sonicated to dissolve all the solids. (Alternatively, the RTX may be initially dissolved in a small volume of ethanol or DMSO, and this solution may then be added to the aqueous component.) The full mixture in the flask is diluted to volume (100.00 ml) with water and this is mixed by an inversion process. The full formulation is filtered through a 0.2 μm PTFE filter.

A formulation according to Formulation 11 is prepared using 200 mcg RTX, 300 mcg Polysorbate 80 (using commercially available polysorbate 80); 5.4 mg of sodium chloride, 500 mcg of dextrose, 1.38 mg sodium phosphate monobasic monohydrate, 2.84 mg sodium phosphate dibasic anhydrous, and water to 1 mL, then pH is adjusted with hydrochloric acid/sodium hydroxide to 7.2. As noted above, the dextrose may be omitted.

A formulation according to Formulation 13 is prepared using 25 mcg RTX, 30 mg Polysorbate 80 (using commercially available polysorbate 80); 5.4 mg of sodium chloride, 50 mg of dextrose, 1.38 mg sodium phosphate monobasic monohydrate, 2.84 mg sodium phosphate dibasic anhydrous, water to 1 mL, then pH is adjusted with hydrochloric acid/sodium hydroxide to 7.2. As noted above, the dextrose may be omitted.

Further details on techniques for formulation and administration may be found in Gennaro, A., Ed., Remington's Pharmaceutical Sciences, 18th Ed. (1990) (Mack Publishing Co., Easton, Pa.).

### Dosage

The methods described herein are for use with any subject in whom RTX is effective, e.g., able to bind and activate TRPVI or a homolog thereof, and who is in need of treatment for inflammatory dysfunction of the oral mucosa and/or neurogenic inflammation. In embodiments, the RTX is administered at a dose of 0.1-150 μg. In embodiments, the dose of RTX ranges from 0.1-0.5 μg, 0.5-1 μg, 1-2 μg, 2-5 μg, 5-10 μg, 10-20 μg, 20-30 μg, 30-40 μg, 40-50 μg, 50-60 μg, 60-70 μg, 70-80 μg, 80-90 μg, 90-100 μg, 100-110 μg, 110-120 μg, 120-130 μg, 130-140 μg, or 140-150 μg.

In embodiments, the RTX is administered in one dose. In embodiments, the RTX is administered in repeated doses. In embodiments, the RTX is administered in 1, 2, 3, 4, or 5 doses.

In embodiments, the RTX is administered daily. In embodiments, the RTX is administered every other day. In embodiments, the RTX is administered weekly.

### Neurogenic Inflammation and Pain

Inflammatory dysfunction of the oral mucosa is associated with neurogenic inflammation and pain. In embodiments inflammatory dysfunction of the oral mucosa is associated with neurogenic inflammation. In embodiments inflammatory dysfunction of the oral mucosa is associated with pain.

In embodiments, the pain may be reduced using compositions and methods described herein. In embodiments, the pain may be abrogated using compositions and methods described herein. In embodiments, the pain is orofacial pain.

In embodiments, the pain is attributed to disorders of dentoalveolar structures, cancer, neuropathic pain, or idiopathic pain. In embodiments, the pain is attributed to disorders of dentoalveolar structures. In embodiments, the pain is attributed to cancer. In embodiments, the pain is attributed to neuropathic pain. In embodiments, the pain is attributed to idiopathic pain. In embodiments, the pain is attributed to stomatitis. In embodiments, the pain is attributed to caudal stomatitis. In embodiments, the pain is attributed to chronic gingivostomatitis (CGS). In embodiments, the pain is attributed to feline chronic gingivostomatitis (FCGS). In embodiments, the pain is attributed to radiotherapy. In embodiments, the pain is attributed to dental pain or oral mucosal pain. In embodiments, the pain is attributed to dental pain. In embodiments, the pain is attributed to oral mucosal pain. In embodiments, the pain is attributed to oral mucosal inflammation.

In embodiments, cancer is treated with radiotherapy. In embodiments the pain is attributed to radiotherapy or chemotherapy induced mucositis.

In embodiments, the idiopathic pain is attributed to burning mouth syndrome.

### EXAMPLES

### 1. Preliminary Safety Study of Oral Topical Application of Resiniferatoxin (RTX) for the Control of Pain Associated with Feline Chronic Gingivostomatitis (FCGS)

This example provides safety data related to oral topical application of RTX. The primary objective of this study was to determine the initial tolerance of RTX when it is topically administered under anesthesia to the oral cavity of cats as a single treatment.

Four cats were enrolled in this study. Each cat presented with mild to moderate gingival inflammation. The cats were allocated to two treatment groups according to severity of gingival inflammation. Group 1 cats, those with mild cases of gingivitis, were dosed on study Day 0 with a diluted RTX solution (12.5 μg/mL) applied topically to the maxillary and mandibular gingival tissue (buccal and palatal/lingual gingiva). Group 2 cats, those with current or recent historical moderate gingivitis, underwent the same treatment regimen on study Day 0 with the addition of application to the palatal and sublingual tissue.

Total dose of RTX tested per cat (diluted with sterile saline for a total volume of 2 mL) was: 17.5 μg (Group 1 cat), 23.75 μg (Group 1 cat), or 25 μg (Group 2 cats) RTX. One cat (pudding) received 17.5 μg RTX, one cat (Clank) received 23.75 μg RTX, and two cats (Forbes and Bianca) received 25 μg RTX. A single application of RTX was topically applied inside the mouth of each cat. The RTX was applied to the entire mouth.

Cats were anesthetized during treatment administration and for 30 minutes post-dose. Diphenhydramine (2 mg/kg, IM) was given as an anesthetic pre-treatment a minimum of 30 minutes prior to dosing. Induction was then performed using a combination of medetomidine 0.03 mg/kg, ketamine 5 mg/kg and butorphanol 0.2 mg/kg; dose volume 0.1 mL/kg, IM. Following application of a lidocaine hydrochloride spray to the larynx, intubation was performed and anesthesia was maintained using an isofluraneoxygen mixture. Standard anesthetic monitoring of vital signs was performed approximately every 5-10 minutes following dosing and precautions were taken to avoid low body temperature throughout the procedure. Cats remained under anesthesia for 30 minutes following dosing for additional monitoring. Upon removal from anesthetic, reversal drug atipamezole (0.2 mg/kg, IM) was administered.

To assess RTX tolerance, adverse event monitoring was performed using the following parameters: measurement of vital signs during anesthesia; physical examinations performed 0.25, 0.5, 1, 2, 3 and 4 hours following recovery from anesthesia (i.e. removal of endotracheal tube) as well as 1, 3 and 7 days post-dose in both the morning (AM) and afternoon (PM); twice daily clinical observations; daily food consumption measurement.

Following RTX application, intermittent lip smacking or lip licking were noted in both groups (Pudding and Clank, Group 1; Bianca and Forbes, Group 2), as was salivation (Pudding and Clank, Group 1; Bianca, Group 2). Onset of these observations ranged from 15 minutes to 2 hours following recovery from anesthesia and all were resolved by the day following dosing. Additional findings included mild increases in the severity of gingivitis in 1 cat from each treatment group (Pudding, Group 1; Forbes, Group 2) as well as a moderate increase in severity from 1 cat (Bianca) in Group 2 who also presented with redness of the lips. Observation of changes in severity of gingivitis were noted between 15 minutes following recovery from anesthesia and 1-day post-dose. Both cases of mild changes resolved by 3 days post-dose without intervention. The cat (Bianca) showing a moderate increase in gingival inflammation (scores of moderate and marked compared to mild at baseline), returned to a moderate classification which continued through study conclusion.

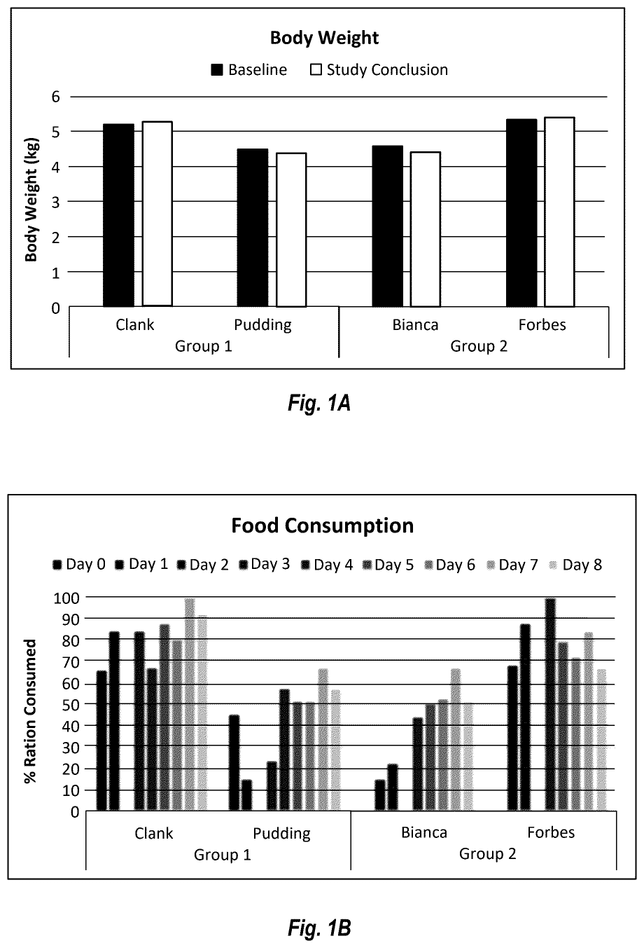

The cats were followed for 8 days following administration of RTX to assess tolerability and safety of RTX. Cats' food consumption over the 8 days following administration of RTX is shown in FIG. 1B. Although there appears to be a transient decrease in food consumption, the cats' food consumption returned to normal by day seven. The cats' body weight was determined before administration of RTX and 8 days after administration of RTX. FIG. 1A shows that despite the transient decrease in food consumption, by day 8 the weight was back to normal (i.e. weight before administration of RTX).

This study demonstrated that RTX was well tolerated with only mild and transient adverse events such as: transient lip licking/Redness of the lips, transient salivation (15 min to 2 hrs. following administration), mild increase in the severity of the gingivitis (up to 24 hrs.), and transient decrease in food consumption.

### 2. Effectiveness and Safety of Oral Topical Application of Resiniferatoxin (RTX) for the Control of Pain Associated With Feline Chronic Gingivostomatitis (FCGS)

This example provides effectiveness and safety data related to oral topical application of RTX for the control of pain associated with feline chronic gingivostomatitis (FCGS). Effects of RTX treatment on inflammation attributed to feline chronic gingivostomatitis was also observed.

primary objective: demonstrate the effect of RTX in controlling oral pain associated with Feline Chronic Gingivostomatitis (FCGS) in client-owned cats using the Veterinarian Specific Outcome Measures (VSOM) and the Stomatitis Disease Activity Index (SDAI) on Day 28. Characterize the required dose of RTX applied topically to the oral mucosa for the control of pain associated with Feline Chronic Gingivostomatitis (FCGS).

### Inclusion Criteria

Cat had documented diagnosis of FCGS.
Cat was experiencing FCGS clinical signs related to oral pain for at least 3 months following the diagnosis of FCGS.
Oral pain was negatively impacting quality of life.
Medications commonly used for the treatment of pain, except for corticosteroids (see Exclusion Criteria), were acceptable provided they had been administered for at least 2 weeks prior to enrolling in the study, and no changes in regimen were expected while the cat was in the study.
The cat had a composite Veterinarian Specific Outcome Measures (VSOM) (see paragraphs and below) for 3 activities selected by the Investigator of 10 or higher, with each question having a score of at least 3.

### Exclusion Criteria

Cat less than 12 months of age.
Cat's body weight less than 2 kg.
Cat unlikely to survive a 30-min anesthesia or sedation procedure.
Cat was unlikely to survive 4 weeks due to health issues other than oral pain.
Cat was unavailable for the entire study duration or was felt to be unsuitable by the Investigator for another reason.
Cat was participating in another study
Cat was pregnant or lactating.
Cat underwent an oral surgical procedure within 30 days prior to screening.
Cat received cannabinoid agonists such as cannabidiol (CBD) or anandamide within 28 days of starting the study.
Cat received oral corticosteroids within 1 week of starting the study, or injectable corticosteroids within 6 weeks of starting the study, and/or required medication or supplements during the study that may have interfered with the objective of the study.

Twenty-eight cats, each weighing over 2 kg and over 12 months of age, were enrolled in this study. There was no apparent trend in the ages of cats enrolled in the study. Enrolled cats were physically examined (their physical exam findings such as general appearance, ears/eyes/mouth, abdominal/gastrointestinal, musculoskeletal, neurological urogenital, lymphatic etc. . . . were recorded). Their weight and vital signs were recorded as well.

### Effectiveness Variables

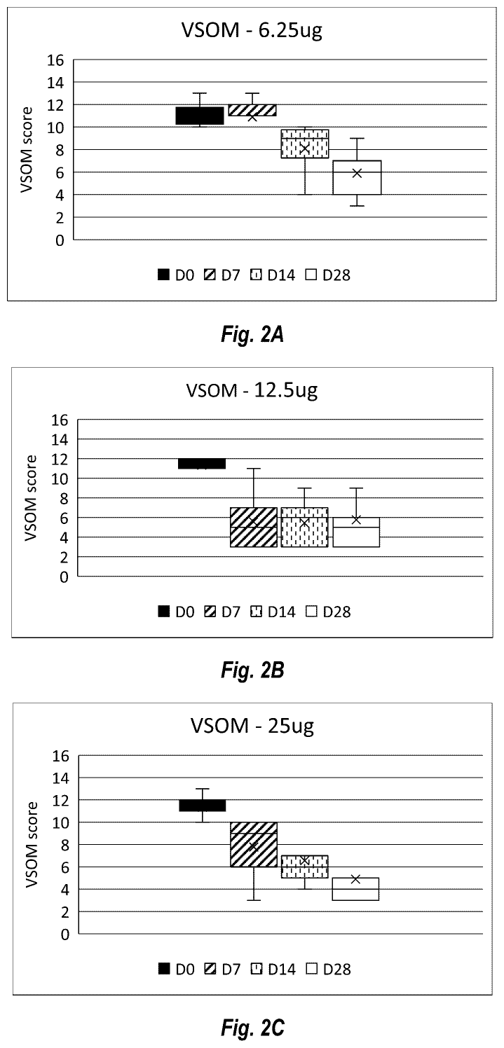

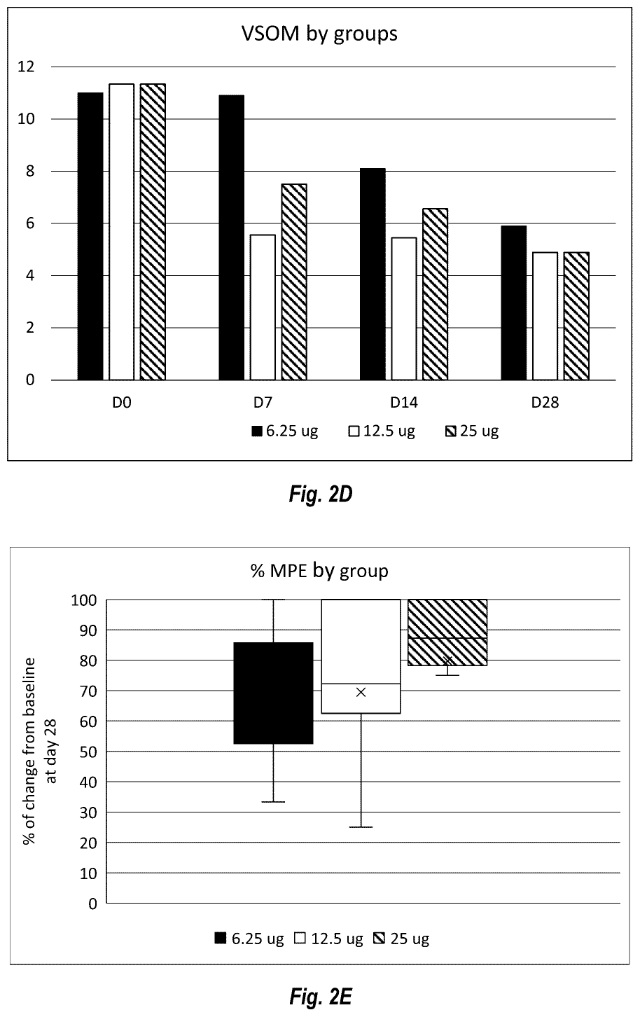

Veterinarian Specific Outcome Measures (VSOM)—Qualitative assessment of common stomatitis signalment was carried out prior to the beginning of treatment and on day 7, 14, and 28 (at the conclusion of treatment). Cats were assessed for drooling, pain while eating, pain at manipulation and swelling of the oral tissue. Each clinical sign was graded from 1 to 5 (1=no problem; 5=Impossible). Results are presented in FIGS. 2-4.

Treatment success was defined as “a reduction of at least 2 in total VSOM score at Day 28 compared to VSOM score at Day 0. A decrease of less than 2, no change, or an increase in total score was defined as treatment failure. Cats presenting an increase in any individual VSOM were considered a treatment failure regardless of total VSOM score. Based on this definition, the success rate on day 28 was 90.0% for Cohort 1 (6.25 μg RTX), 80.0% for Cohort 2 (12.5 μg RTX), and 88.9% for Cohort 3 (25 μg RTX). The results of each cohort were not compared statistically; only descriptive statistics were generated due to the small number of cats per cohort and lack of randomization. However, there was no evidence of a relationship between treatment success and dose.

FIG. 2A shows VSOM scores, taken at day 0 (before the beginning of treatment) day 7, day 14, and day 28. The graphs demonstrate the effectiveness of 6.25 μg, 12.5 μg, and 25 μg RTX treatment. FIG. 2B shows those same VSOM scores organized by treatment group (6.25 μg, 12.5 μg, or 25 μg RTX), demonstrating that similar changes are observed at day 28 in all groups. Finally, FIG. 2C shows % change in VSOM scores from day 0 (before treatment) to day 28. This graph shows the % Maximum possible effect (% MPE), demonstrating that MPE at day 28 ranges from 68% to 80%.

Stomatitis Disease Activity Index (SDAI)—This semi-quantitative assessment of oral inflammation was carried out prior to the beginning of treatment (day 0) and at day 28 (at the conclusion of treatment). Cats were assessed by owner for cat's interest in food and ability to eat, perceived comfort, grooming behavior, and activity levels. Each assessment was graded from 0 to 3 (0=no problem; 3=severe problems). Specifics of the assessment are shown in Table 1 below.

TABLE 1
Owner Evaluation Questions

| | |
|---|---|
| Appetite: | 3 = eats only pureed food, or only when hand fed |
| | 2 = eats wet food; cannot eat dry food |
| | 1 = eating wet and dry food, but less than normal amount |
| | 0 = eating normally |
| Activity | 3 = no interest in people or other pets, spends most of time |
| Level: | sleeping |
| | 2 = low activity level, but will play occasionally when |
| | engaged by people or other pets |
| | 1 = plays spontaneously, but not frequently |
| | 0 = normal activity level (playful and active) |
| Grooming | 3 = will not groom |
| Behavior: | 2 = grooms occasionally but not at ‘pre-illness’ level |
| | 1 = grooming excessively |
| | 0 = grooming normally |
| Perceived | 3 = severe discomfort |
| Comfort: | 2 = moderate discomfort |
| | 1 = mild discomfort |
| | 0 = most comfortable |

The average of the scores in Table 1 (total score divided by 4) were entered in the “owner evaluation” box on the initial SDAI (Stomatitis Disease Activity Index) tabulation sheet of Table 2. The rest of the questions on SDAI survey were completed by the veterinarian prior to the beginning of treatment (day 0) and at day 28 (at the conclusion of treatment). Each assessment was graded from 0 to 3 (0=no problem; 3=severe problems) as follows:

| | |
|---|---|
| Weight (compared with most | 0 = gain >0.5 kg |
| recent visit): | 1 = gain >0.25 kg but <0.5 kg |
| | 2 = <0.25 kg gain |
| | 3 = weight loss |

| | |
|---|---|
| Inflammation (specified sites | 0 = normal tissue |
| as graded by clinician) | 1 = mild inflammation or ulceration |
| | 2 = moderate inflammation or ulceration |
| | 3 = severe inflammation or ulceration |

TABLE 2
Stomatitis Disease Activity Index - SDAI

| | | | | |
|---|---|---|---|---|
| Stomatitis Disease Activity Index | 0 | 1 | 2 | 3 |

| | |
|---|---|
| 1. | Owner evaluation |
| 2. | Weight (compared to Day 0) |
| 3. | Maxillary buccal mucosal inflammation |
| 4. | Mandibular buccal mucosal inflammation |
| 5. | Maxillary attached gingival inflammation |
| 6. | Mandibular attached gingival inflammation |
| 7. | Inflammation lateral to palatoglossal folds |
| 8. | Molar salivary gland inflammation |
| 9. | Oropharyngeal inflammation |
| 10. | Lingual and/or sublingual inflammation |

TOTAL SCORE (max = 30)

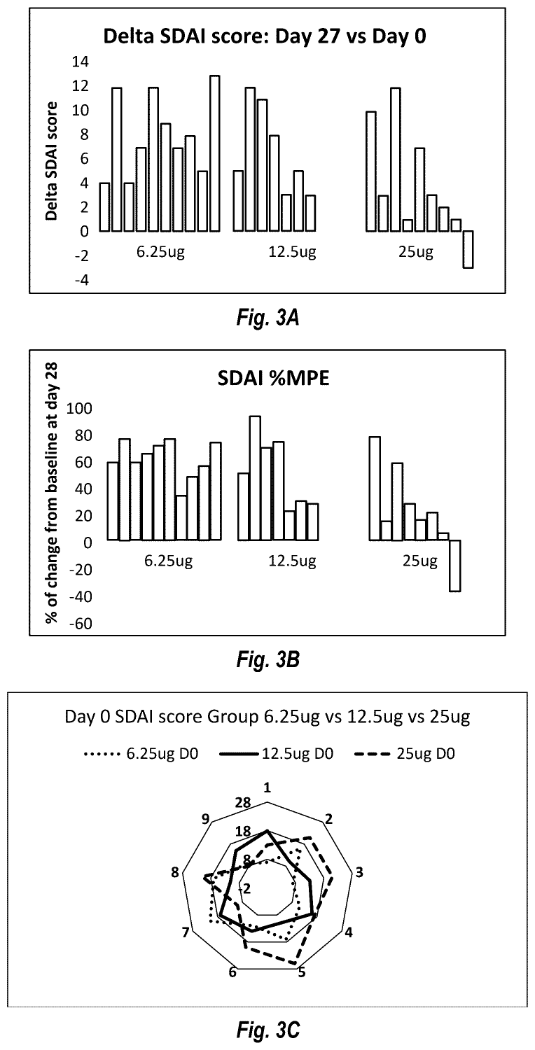

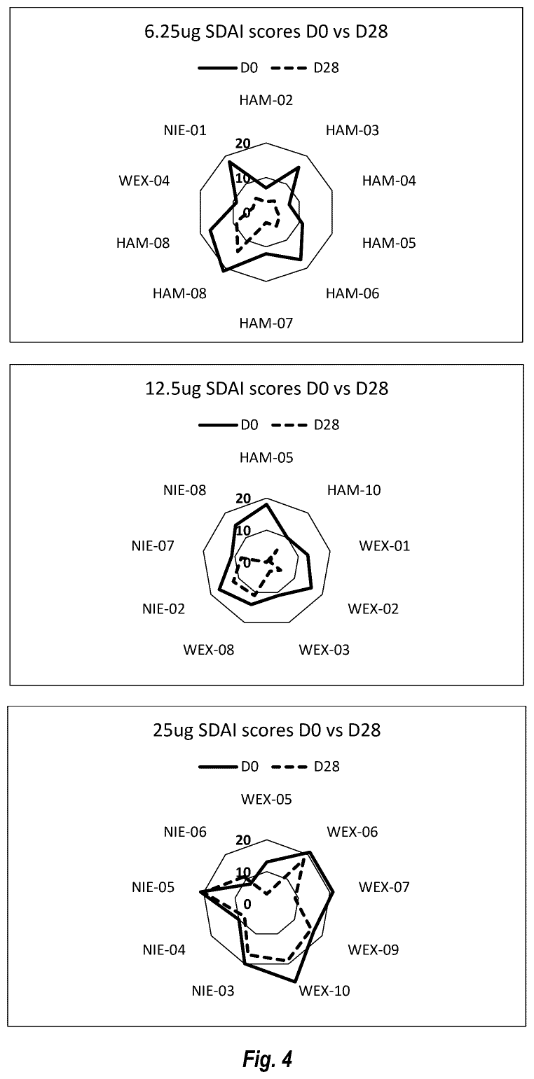

FIG. 3A shows the change in SDAI scores for each cat, taken before the beginning of treatment (day 0) and on day 28. FIG. 3A demonstrates the effectiveness of 6.25 μg, 12.5 μg, and 25 μg RTX treatment even at the lowest amount used 6.25 μg. FIG. 3B shows % change in SDAI scores from day 0 (before treatment) to day 28. This graph shows the % Maximum possible effect (% MPE), demonstrating that MPE at day 28 ranges from 30% to 85% (with most cats experiencing improvement of about 60%). FIG. 3C shows the baseline SDAI score (day 0) for each cat in three treatment groups (6.25 μg, 12.5 μg, and 25 μg RTX). FIG. 4 shows the baseline SDAI score (day 0) for each cat in the three treatment groups (6.25 μg, 12.5 μg, and 25 μg RTX) and the SDAI score on day 28. FIG. 4 shows smaller trend of improvement at higher doses (25 μg) of RTX and largest trend of improvement at 12.5 μg.

For SDAI scores (FIGS. 3A-3B and FIG. 4) smaller trend of improvement was observed at higher doses. FIG. 3C shows that coincidentally, the group treated with the highest dose (25 μg RTX) had the highest SDAI score pretreatment. Thus, it is possible that severity of pretreatment inflammation conditions the overall response to treatment. The treatment being most effective for moderate disease.

Treatment success using the SDAI metric was defined post-hoc as a 20% reduction in the Day 28 SDAI compared to Day 0. Based on this definition, the percentage success was 100.0%, 80.0% and 44.4% for Cohorts 1, 2, and 3 respectively. The discrepancy in the success rate of Cohort 3 using the two metrics may be due to a higher baseline SDAI score. However, these results confirmed that an increase in inflammation as measured by the SDAI did not correlate to treatment success using a behavior metric like the VSOM.

### Safety Variables

Mortality—No mortality, whether by natural death or euthanasia, occurred on this study.

### Adverse Events and Serious Adverse Events—

An adverse event (AE) is defined as any undesirable event, expected or not, occurring to a study animal during the study, regardless of whether the event was considered to have a causal relation to a study treatment. Adverse events were reported at scheduled times during the study from Day 0 to the last day of the study. Certain adverse events, defined as serious adverse events, were reported in an expedited fashion to allow for possible study protocol modifications. A Serious Adverse Event (SAE) is defined as any medical occurrence that results in hospitalization longer than 24 hours or results in significant disability or morbidity or is life threatening or results in death.

One SAE occurred during the in-life of this study, determined as medically significant by the Investigator. On Study Day 14, NIE-08 (Cohort 2; 6.25 μg/mL topical resiniferatoxin dose of 12.5 μg) was reported as being extremely painful, which the Investigator determined as possibly being related to stress from travel or mouth manipulation. The cat had an increase in screaming episodes and was unable to eat food that was not pureed. The event was deemed by the Investigator to be serious on Study Day 14 and was noted to be improving on Study Day 17 when the patient was permanently removed from the study. However, this cat was included in the effectiveness analysis as treatment failure. HAM-01 (Cohort 1; 3.12 μg/mL topical resiniferatoxin dose of 6.25 μg) developed respiratory signs by day 9 unrelated to RTX application and was removed from the study on day 9. WEX-09 (Cohort 3; 12.5 μg/mL topical resiniferatoxin dose of 25 μg) was removed from the study due to progressive pain documented as SAE and was included in the effectiveness analysis as treatment failure.

Adverse events were described by the Investigator according to the following descriptions:
Mild; asymptomatic or mild symptoms; clinical signs or diagnostic observations only; intervention not indicated.
Moderate; minimal, outpatient or non-invasive intervention indicated; moderate limitation of activities of daily living (ADL).
Severe or medically significant but not immediately life-threatening; hospitalization or prolongation of hospitalization indicated; disabling; significantly limiting ADL.

Clinical signs definitively related to progression in pain were not considered adverse events unless believed to be associated with RTX administration. Patients experiencing adverse events received appropriate medical therapy, as determined by the Investigator.

Severity, causality and outcome of adverse events were recorded. Causality is a determination of whether there was a reasonable possibility that the drug may have caused or contributed to an AE. This relationship was described as “unlikely”, “possible”, “probable”, “definite”, or “unknown.”

Blood (4-5 mL) was collected at the beginning of the study for complete blood count (CBC) and serum biochemistry panel.

Most of the AE were graded as mild. There were no consistent trends evident between AE and dose, severity, or causality. There were increases in the incidence of AE on Study Days 7 and 14, which coincided with study visits, but this had decreased on Study Day 28.

### Sedation and Anesthesia

Fasted cats were pretreated with antihistaminic drug at least 30-45 minutes prior to RTX treatment (antihistamine was used to counter possible allergic reaction cats and dogs have to polysorbate 80). Enrolled cats were anesthetized using standard feline sedation/anesthesia based on a mix of analgesics/sedatives/anesthetics. The cats were monitored by auscultation and chest movement observation. Heart rate and oxygenation were monitored using a pulse oximeter throughout the duration of procedure. Blood pressure was monitored using an oscilometric non-invasive blood pressure machine (e.g., tail cuff and or limb). Body temperature (98 to 99° F.) was maintained using a heated water blanket or equivalent thermal barrier (e.g., Vetko). A balanced IV solution could be used to maintain adequate hydration (approximately 2 mL/kg/hr intravenous (IV) infusion) and/or fluid bolus (2-5 mL/kg IV), as needed. After the cat reached an adequate anesthesia level, the RTX solution was applied. A copy of the Anesthesia Record was included in the study file.

Prior to treatment with RTX, the location of the oral lesion(s) was documented, and a photograph of the oral cavity was taken to document the oral lesions before treatment and on Day 28.

### RTX Preparation and Application

RTX was provided as a 25 μg/mL (2.4 mL) solution in a 2R glass vial. The IVP was maintained in its original sterile glass container (vial) until use. IVP was brought to room temperature prior to administration and was gently swirled but not shaken prior to use. Dose and preparation instructions are described in Table 3.

Each animal was randomly assigned to one of three groups. Ten cats (group 1) were treated with 6.25 μg RTX (total) diluted with sterile saline for a total volume of 2 mL. Nine cats (group 2) were treated with 12.5 μg RTX (total) diluted with sterile saline for a total volume of 2 mL. Nine cats (group 3) were treated with 25 μg RTX (total) diluted with sterile saline for a total volume of 2 mL.

TABLE 3
Animal treatment - Topical Oral Application

| | | Total | Total | Total | | | | |
|---|---|---|---|---|---|---|---|---|
| | | RTX | RTX | Saline | Total | | | |
| | | Dose | vol | vol | Vol | Concentration | | |
| Cohort | n | (μg) | (mL) | (mL) | (mL) | (μg/mL) | Dilution Instructions | |
| 1 | 10 | 6.25 | 0.25 | 1.75 | 2.0 | 3.12 | 1. | Place 0.25 mL of RTX solution in a |
| | | | | | | | | labelled 5 mL conical plastic tube. |
| | | | | | | | 2. | Add 1.75 mL of sterile saline. |
| | | | | | | | 3. | Gently swirl the solution to create 2 mL |
| | | | | | | | | of the final concentration RTX solution. |
| 2 | 9 | 12.5 | 0.5 | 1.5 | 2.0 | 6.25 | 1. | Place 0.5 mL RTX solution in a |
| | | | | | | | | labelled 5 mL conical plastic tube. |
| | | | | | | | 2. | Add 1.5 mL of sterile saline. |
| | | | | | | | 3. | Gently swirl the solution to create 2 ml |
| | | | | | | | | of the final concentration RTX solution. |
| 3 | 9 | 25 | 1 | 1 | 2.0 | 12.5 | 1. | Place 1 mL RTX solution in a labelled |
| | | | | | | | | 5 mL conical plastic tube. |
| | | | | | | | 2. | Add 1 mL of sterile saline. |
| | | | | | | | 3. | Gently swirl the solution to create 2 ml |
| | | | | | | | | of the final concentration RTX solution. |

RTX solution was applied using a clean cotton tip applicator directly to the visible lesions and adjacent tissue. The non-target dosing areas (caudal mouth/pharynx) were protected using a 4×4 gauze. Any targeted area was dried with sterile gauze or using the air-water syringe system. The saturated applicator for was gently pressed along the targeted areas for 10 seconds. Two to three coats per lesion were applied using the same cotton tip applicator. The area was allowed to dry for a few seconds before moving to the next lesion by exposing the area to the air or by gently using the air-water syringe system. When possible, and in absence of active bleeding due to manipulation, the RTX solution was allowed to drip into the gingival sulcus. If a target area was actively bleeding, an attempt to decrease or stop the bleeding was made before the RTX application.

After the treatment of all lesions, any remaining RTX solution was applied to the adjacent tissue of the most affected areas. Once all applied RTX had dried for at least 20 min, the treated areas were gently cleaned with a 4×4 gauze lightly moistened with saline.

After oral topical application of RTX, cats remained under general anesthesia for at least 10 minutes (for a minimum total anesthesia time of 30 minutes).

If used, reversal drugs such as atipamezole were discouraged for at least 1 hour after application of RTX solution. If an acute reaction occurs (e.g., involuntary motor movement or respiratory effort, exacerbated cardiovascular changes, nociceptive response) during the administration, cats may receive short-lasting analgesic such as, but not limited to fentanyl (1-2 μg/kg IV) or sufentanil, and/or propofol (0.5 mg/kg IV) to control motor movement as needed. The characteristics and magnitude of these reactions are reported as adverse event (AE).

If anaphylactic-like reactions (e.g., facial edema, auricular edema, excessive airway secretion, tachycardia, hypotension, urticaria, vomiting/regurgitation, diarrhea, erythema and/or elevated temperature) were observed during treatment or recovery, and if the cat was unstable and in a life-threatening state, dexamethasone sodium phosphate at 0.2-0.4 mg/kg was IV administered over 1 minute. The continuation in the study of any cat having received corticosteroids under the conditions described above was re-evaluated due to their potent anti-inflammatory effect. All drugs used to treat adverse events post-administration of RTX are reported.

### Clinical Observations Post-treatment

After treatment was completed, up to 3 days of a short acting analgesic such as, but not limited to, buprenorphine was prescribed for control of pain related to treatment as needed. The administration of buprenorphine decreased the number of adverse reactions. Cats in Cohort 1 were not treated with buprenorphine following administration of RTX. Cats in Cohorts 2 and 3 were treated with buprenorphine following administration of RTX.

On Day 0, starting once the RTX was applied to all oral lesions (time 0), clinical observations were carried out by the veterinarian at 10, 30, 45, 60 (±5 minutes), as well as at 2 hours (±10 minutes) and 4 hours (±15 minutes), post-treatment administration. Cats were observed past 4 hours post-treatment administration, as necessary or until resolution of abnormal observations.

Clinical signs such as, but not limited to, panting, hypersalivation, tachycardia, restlessness had been reported as side effects after administration of RTX in cats. These signs were resolved within approximately 3 hours post-treatment without the need of medical intervention.

### Summary of Results

Veterinary specific outcome measurement (VSOM) results—on Day 7, Cohorts 2 and 3 had a higher precent success than Cohort 1, which may have been influenced by the use of post-treatment buprenorphine in these two cohorts. However, the percent success of Cohort 1 kept increasing and by Day 28, this cohort had the highest precent success. On Day 14, all cats in Cohort 2 were a success, but 2 of them had failed by Day 28. Treatment success (as defined above in section on Effectiveness Variables-VSOM) on day 28 was 90.0% for Cohort 1 (6.25 μg RTX), 80.0% for Cohort 2 (12.5 μg RTX), and 88.9% for Cohort 3 (25 μg RTX).

Stomatitis disease activity index (SDAI) results—a case was considered a success if the total SDAI score has decreased by 20% or more between Day 0 and Day 28. An inverse relationship was observed between dose and the percentage of success with the highest success in Cohort 1 (6.25 μg RTX) and lowest in Cohort 3 (25 μg RTX). Treatment success was 100.0%, 80.0% and 44.4% for Cohorts 1, 2, and 3 respectively.

However, as seen in Table 4, the average total SDAI scores on Day 0 were different across treatment cohorts with the highest baseline SDAI in Cohort 3. Cats were not randomized to cohorts and the variability in the baseline SDAI scores should be considered when interpreting the percent success and failure based on a >20% decrease in SDAI scores between Day 0 and Day 28.

TABLE 4
SDAI Scores: Average Cohort Total Scores on Day 0

| Day 0 | Average SDAI total score |
|---|---|
| Cohort 1 | 13.50 |
| Cohort 2 | 13.20 |
| Cohort 3 | 17.44 |

Comparison of VSOM and SDAI effectiveness assessments: There was a substantial discrepancy in the percentage of success using the VSOM vs SDAI metrics for Cohort 3 due to the higher baseline SDAI. However, this discrepancy confirmed that the degree of inflammation did not always correlate with a positive response to treatment, which was assessed behaviorally in the VSOM evaluation. Because the primary goal of RTX application is to provide oral pain relief to encourage eating and improve quality of life, rather than reduce inflammation, VSOM was determined to be the superior metric in this study.

Cats were weighed on days 0, 7, 14, and 28. Normal body weights were reported for 24 of the 28 cats. Two cats (one in Cohort 1 and one in Cohort 3) had abnormal (low) weight on day 0, but normal weight by day 28. One cat (Cohort 1) exhibited weight loss on day 28 compared to day 0. One cat (Cohort 2) exhibited weight loss on day 14.

Cats' ability to eat was assessed on days 0, 7, 14, and 28. The majority of the cats' ability to eat improved throughout the study. On day 28, no differences in ability to eat were detected between cohorts. Overall, most of the cats in all cohorts that completed the study had an improvement in the ability to eat, with only one cat (in Cohort 2) getting worse.

### Conclusions

Even low concentration of RTX (6.25 μg) is effective in controlling pain and inflammation for 28 days.

The application of RTX on inflamed and fragile oral mucosa appears to be safe as cats did not exhibit signs of systemic RTX exposure.

The study demonstrated that buprenorphine administered up to 72 hours post treatment improved the tolerability of RTX treatment

Adverse events are mostly related to post-treatment discomfort which is manageable with standard pain therapy (Buprenorphine administered once a day for 3 days). The most common AEs were weight loss, decreased appetite, hypersalivation, decreased ability to eat and painful/oral pain.

Body weight was not adversely impacted by RTX treatment in most cats. No information was obtained regarding safe maintenance of hot sensations.

The study demonstrated a high success rate in all cohorts using the VSOM metric and confirmed that a behavior metric like VSOM was superior to the SDAI which also measured inflammation. The safety profile for this route of administration was acceptable as most of the AEs were considered to be mild in severity and the cats did not show signs of systemic exposure to RTX.

The complete disclosures of all publications cited herein are incorporated herein by reference in their entireties as if each were individually set forth in full herein and incorporated.

Various modifications and alterations to the embodiments disclosed herein will become apparent to those skilled in the art without departing from the scope and spirit of this disclosure. Illustrative embodiments and examples are provided as examples only and are not intended to limit the scope of the present invention.

1. A method for treating an inflammatory dysfunction of the oral mucosa, comprising topically administering to a subject in need of treatment of the inflammatory dysfunction of the oral mucosa a therapeutically effective amount of resiniferatoxin (RTX).

2. A composition comprising resiniferatoxin (RTX) for use in a method for treating an inflammatory dysfunction of the oral mucosa, the method comprising topically administering to a subject in need of treatment of the inflammatory dysfunction of the oral mucosa a therapeutically effective amount of said composition.

3. The method of claim 1 or composition for use of claim 2, wherein the inflammatory dysfunction of the oral mucosa is associated with neurogenic inflammation.

4. The method or composition for use of claim 3, wherein the neurogenic inflammation is reduced.

5. The method of claim 1 or composition for use of claim 2, wherein the inflammatory dysfunction of the oral mucosa is associated with pain.

6. The method or composition for use of claim 5, wherein the pain is reduced.

7. The method or composition of claim 5, wherein the pain is orofacial pain.

8. The method or composition for use of claim 3, wherein the subject is a mammal.

9. The method or composition for use of claim 8, wherein the mammal is a human.

10. The method or composition for use of claim 8 wherein the mammal is a cat, dog, horse, pig, ruminant, cow, sheep, goat, or domesticated mammal.

11. The method or composition for use of any one of the preceding claims, wherein the RTX is administered in a dose of from about 0.1 μg to about 150 μg.

12. The method or composition for use according to any one of the preceding claims, wherein the method comprises administering a pharmaceutical formulation comprising the RTX and a pharmaceutically acceptable carrier.

13. The method or composition for use of claim 12, wherein the pharmaceutically acceptable carrier comprises water.

14. The method or composition for use of claim 12 or 13, wherein the pharmaceutically acceptable carrier comprises polysorbate 80.

15. The method or composition for use of any one of claims 12-14, wherein the pharmaceutically acceptable carrier comprises polyethylene glycol.

16. The method or composition for use of any one of claims 12-15, wherein the pharmaceutically acceptable carrier comprises a sugar or sugar alcohol.

17. The method or composition for use of any one of claims 12-16, wherein the pharmaceutically acceptable carrier comprises mannitol.

18. The method or composition for use of any one of claims 12-17, wherein the pharmaceutically acceptable carrier comprises dextrose.

19. The method or composition for use of any one of claims 12-18, wherein the pharmaceutically acceptable carrier comprises a pharmaceutically acceptable buffer, optionally wherein the pharmaceutically acceptable buffer is phosphate buffer and/or the pH of the formulation is about 6.0-7.6 or about 7.2.

20. The method or composition for use of any one of claims 12-19, wherein the pharmaceutically acceptable carrier comprises a pharmaceutically acceptable salt.

21. The method or composition for use of any one of claims 12-20, wherein the pharmaceutically acceptable salt is NaCl.

22. The method or composition for use of any one of claims 12-21, wherein the concentration of RTX in the pharmaceutical formulation is in the range of 0.02 to 300 μg/ml.

23. The method or composition for use of claim 22, wherein the concentration of RTX in the pharmaceutical formulation is in the range of 0.02-0.1 μg/ml, 0.1-1 μg/ml, 1-5 μg/ml, 5-10 μg/ml, 10-20 μg/ml, 20-50 μg/ml, 50-100 μg/ml, 100-150 μg/ml, 150-200 μg/ml, 200-250 μg/ml, or 250-300 μg/ml.

24. The method or composition for use of claim 22, wherein the concentration of RTX in the pharmaceutical formulation is in the range of 0.1-50 μg/ml.

25. The method or composition for use of any one of the preceding claims, wherein the RTX is topically administered in a volume of 0.5-5 ml, optionally wherein the volume is in the range of 0.5-1.0 ml, 1.0-1.5 ml, 1.5-2 ml, 2-3 ml, 3-4 ml, or 4-5 ml.

26. The method or composition for use of any one of the preceding claims, wherein the method further comprises administering a general or a local anesthetic prior to administration of RTX.

27. The method or composition for use of any one of the preceding claims, wherein the method further comprises administering an analgesic following administration of RTX.

28. The method or composition for use of claim 27, wherein the analgesic is an opioid or a nonsteroidal anti-inflammatory drug (NSAID).

29. The method or composition for use of any one of the preceding claims, wherein the pain is attributed to disorders of dentoalveolar structures, cancer, neuropathic pain, or idiopathic pain.

30. The method or composition for use of claim 29, wherein the pain is attributed to neuropathic pain.

31. The method or composition for use of claim 29, wherein the pain is attributed to idiopathic pain.

32. The method or composition for use of claim 31, wherein the idiopathic pain is attributed to burning mouth syndrome.

33. The method or composition for use of any one of the preceding claims, wherein the pain is attributed to stomatitis.

34. The method or composition for use of any one of the preceding claims, wherein the pain is attributed to chronic gingivostomatitis (CGS).

35. The method or composition for use of claim 34, wherein the chronic gingivostomatitis is feline chronic gingivostomatitis (FCGS).

36. The method or composition for use of any one of the preceding claims, wherein the pain is attributed to cancer.

37. The method or composition for use of any one of the preceding claims, wherein the pain is attributed to radiotherapy.

38. The method or composition for use of any one of the preceding claims, wherein the pain is attributed to disorders of dentoalveolar structures.

39. The method or composition for use of claim 38, wherein the pain is attributed to dental pain or oral mucosal pain.

40. The method or composition for use of any one of the preceding claims, wherein the pain is attributed to radiotherapy induced mucositis.

41. A method for treating a neurogenic inflammation associated with an inflammatory dysfunction of the oral mucosa, comprising topically administering to a subject in need of treatment of the neurogenic inflammation a therapeutically effective amount of resiniferatoxin (RTX).

42. A composition comprising resiniferatoxin (RTX) for use in a method for treating a neurogenic inflammation associated with an inflammatory dysfunction of the oral mucosa, the method comprising topically administering to a subject in need of treatment of the neurogenic inflammation associated with an inflammatory dysfunction of the oral mucosa a therapeutically effective amount of said composition.

43. The method of claim 41 or composition for use of claim 42, wherein the neurogenic inflammation is reduced.

44. The method of claim 41 or composition for use of claim 42, wherein the neurogenic inflammation is associated with pain.

45. The method or composition for use of claim 44, wherein the pain is reduced.

46. The method or composition for use of claim 44, wherein the pain is orofacial pain.

47. The method or composition for use of claim 44, wherein the pain is attributed to disorders of dentoalveolar structures, cancer, neuropathic pain, or idiopathic pain.

48. The method or composition for use of claim 47, wherein the pain is attributed to neuropathic pain.

49. The method or composition for use of claim 47, wherein the pain is attributed to idiopathic pain.

50. The method or composition for use of claim 49, wherein the idiopathic pain is attributed to burning mouth syndrome.

51. The method or composition for use of claim 44, wherein the pain is attributed to stomatitis.

52. The method or composition for use of claim 44, wherein the pain is attributed to chronic gingivostomatitis (CGS).

53. The method or composition for use of claim 44, wherein the chronic gingivostomatitis is feline chronic gingivostomatitis (FCGS).

54. The method or composition for use of claim 44, wherein the pain is attributed to cancer.

55. The method or composition for use of claim 44, wherein the pain is attributed to disorders of dentoalveolar structures.

56. The method or composition for use of claim 55, wherein the pain is attributed to dental pain or oral mucosal pain.

57. The method or composition for use of claim 44, wherein the pain is attributed to radiotherapy induced mucositis.

58. The method or composition for use according to any one of the preceding claims, wherein the RTX is administered to a plurality of sites.
